(19) United States
(12) Patent Application Publication
(10) Pub. No.: US 2022/0000528 A1
(43) Pub. Date: Jan. 6, 2022

(54) Modular Head Compression Screw System and Device

(71) Applicants: Brandon Lampe, Forney, TX (US); Bryan Duffin, Forney, TX (US); Thomas Zink, San Antonio, TX (US)

(72) Inventors: Brandon Lampe, Forney, TX (US); Bryan Duffin, Forney, TX (US); Thomas Zink, San Antonio, TX (US)

(21) Appl. No.: 17/363,254

(22) Filed: Jun. 30, 2021

Related U.S. Application Data
(60) Provisional application No. 63/046,779, filed on Jul. 1, 2020.

(51) Int. Cl. A61B 17/86

(52) U.S. Cl. CPC A61B 17/861; A61B 2017/681; A61B 17/8695

(57) ABSTRACT

A modular head compression screw system is disclosed and has a cannulated compression screw component with a modular head. The compression screw component includes a tubular body and penetrating tip having a first and second end, and an exterior thread on the tubular body which extends to the second end of the tubular body or penetrating tip. A second exterior thread is positioned around the diameter of the first end to create a posted compression screw. Further, a modular head is removably secured at the first end via the second exterior thread and configured to engage with a driver to advance the compression screw in to the bone. Each modular head component may have a threaded head component which removably secures to the first end of the tubular body, and a fixation component which acts to engage tendons and/or bones.

### CROSS-REFERENCE TO RELATED APPLICATION

The present application claims priority to, and the benefit of, U.S. Provisional Application No. 63/046,779, which was filed on Jul. 1, 2020 and is incorporated herein by reference in its entirety.

### FIELD OF THE INVENTION

The present invention relates generally to a modular head compression screw system for use with surgical procedures such as osteotomies, foot fusions, fractures and other procedures where it is necessary or advisable to use a mechanical fastener as part of the surgery. More specifically, the modular head compression screw system is designed to fit several different head attachments or accessories onto the same post to allow a surgeon or other medical professional to effectively reduce fractures, compress osteotomies and anchor tendons without the need for numerous large surgical sets, pins and the like. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, various types of fasteners are used to secure implants and other devices to bone. In the orthopedic field, screws, pins and other fasteners are used to hold plates, rods or other prosthetic devices in position, or to hold bones or bone fragments in place to facilitate fusion between bone surfaces. Achieving repair across or in the area of a fracture, or fusion between two otherwise unrelated bone segments through the use of a stand-alone screw, is often facilitated by causing compression of the segments to be fused or attached at the cortical surfaces. The prior art has developed a number of screws that generate this kind of compression, as well as plates, pins, accessories or rods which facilitate fusion between bone surfaces. However, small bone surgeons require fasteners and implants that are smaller in size and that are modular to address several solutions to multiple different types of patient needs and medical situations. This area includes bones distal to the elbow or knee that are smaller, more delicate bones than the long bones, and these are bones that function in conjunction with less, more delicate soft tissue in order to achieve movements with greater finesse.

Thus, there is often little bone for purchase, or securing medical devices and even less muscle tissue available to provide a buffer for protruding tips. Consequently, it is imperative in the small bone context that implants are designed to maximize the desired result, with minimum volume. The present invention is designed to provide both a maximum of compression along the longitudinal axis of the screw and secondary fixation and securement solutions. Sometimes a screw has too little fixation to solve the surgical issue, but a full plate provides too much fixation and cannot be adequately secured to the area, further complicating the procedure.

Therefore, there is a long felt need in the art for a modular head compression screw that will provide one system to address several different orthopedic procedures. When a screw has too little fixation to solve an issue, the proposed modular system will incorporate a screw with several different secondary fixation solutions to provide a greater purchase in the bone. Finally, there is a long felt need in the art for a modular head compression screw that is relatively inexpensive to manufacture, and that is both safe and easy to use.

The subject matter disclosed and claimed herein, in one embodiment thereof, comprises a modular head compression screw device and system. The cannulated compression screw component comprises a first and second end with a modular head component positioned at the first end and configured to engage with a driver to advance the screw into the bone. A penetrating tip is positioned at the second end. The modular head component of the cannulated compression screw component further includes several different modular head attachments, which can be secured to and removed from the cannulated compression screw component, depending on the needs or requirements of a user. Therefore, the modular head compression screw system provides a user with the ability to attach different modular heads onto the same post without the need for numerous large surgical sets.

While this specification makes specific reference to the compression screw device of the present invention as a way in which to provide a modular screw head system, it will be appreciated by those of ordinary skill in the art that aspects of the present invention are also equally amenable to other like applications and other such surgical screws and/or surgical issues.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a modular head compression screw comprising a cannulated compression screw component with a modular head. The compression screw component has a tubular body with a first and second end. The tubular body has a tubular wall defining a cavity in which the tubular body is cannulated. The tubular body further includes an exterior thread on an exterior tubular surface portion of the tubular wall which extends to the second end of the tubular body. A second exterior thread is positioned around the diameter of the first end to create a posted compression screw. Further, a modular head is removably secured at the first end via the second exterior thread and configured to engage with a driver to advance the compression screw into the bone. Finally, the tubular body comprises a penetrating tip at the second end. The tubular body and the penetrating tip are formed as a single piece.

In a preferred embodiment, each modular head component comprises a threaded head component and a fixation component. The threaded head component comprises internal threads extending around the circumference which matingly secures to the first end via the second exterior threads of the tubular body. The fixation component acts to engage tendons and/or bones to reduce fractures, compress osteotomies, etc. The fixation component can be positioned radially in any position, and spins independently of the threaded head component.

In another embodiment, the modular head compression screw system comprises a plurality of different modular head configurations. For example, the modular heads can comprise a double winged compression head, a hook plate head, a single wing compression head, a plate head, a simple washer head and combinations thereof. Any other modular head can be utilized as is known in the art, depending on the needs and/or wants of a user.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a side perspective view of one potential embodiment of the tubular body of the modular head compression screw system of the present invention in accordance with the disclosed structure;

FIG. 2 provides a perspective view of one potential embodiment of the double winged compression head of the modular head compression screw system of the present invention in accordance with the disclosed specification;

FIG. 3 depicts a perspective view of one potential embodiment of the hook plate head of the modular head compression screw system of the present invention in accordance with the disclosed structure;

FIG. 4 shows a perspective view of one potential embodiment of the single wing compression head of the modular head compression screw system of the present invention in accordance with the disclosed specification;

FIG. 5 displays a perspective view of one potential embodiment of the plate head of the modular head compression screw system of the present invention in accordance with the disclosed configuration; and

FIG. 6 illustrates a perspective view of one potential embodiment of the simple washer head of the modular head compression screw system of the present invention in accordance with the disclosed structure.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

Generally stated, and in one embodiment thereof, the present invention discloses a modular head compression screw device comprising a cannulated compression screw component with a modular head. The compression screw component includes a tubular body and penetrating tip having a first and second end, and an exterior thread or first thread type on the tubular body which extends to the second end of the tubular body (or penetrating tip) A second exterior thread type is positioned around the diameter of the first end to create a posted compression screw. Further, a modular head is removably secured at the first end via the second exterior thread, and configured to engage with a driver to advance the compression screw component in the bone.

In a preferred embodiment, each modular head component may comprise a threaded head component and a fixation component. The threaded head component includes internal threads extending around the circumference of the body, which matingly secures to the first end via the second exterior threads of the tubular body. The fixation component acts to engage tendons and/or bones to reduce fractures, compress osteotomies, etc. The fixation component can be positioned radially in any position, and spins independently of the threaded head component.

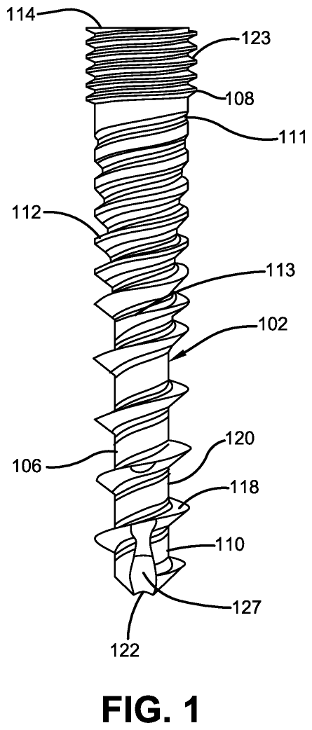

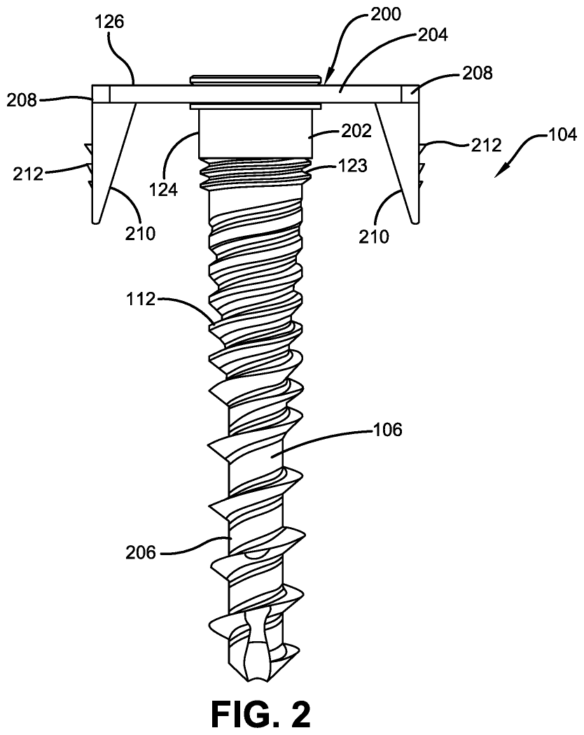

Referring initially to the drawings, FIG. 1 illustrates a modular head compression screw device 100 comprising a cannulated compression screw component 102 with a modular head component 104 (shown in FIGS. 2-6). Both the cannulated screw component 102 and the modular head component 104 may be manufactured using typical machining techniques as are known in the art. Notwithstanding, the cannulated compression screw component 102 and modular head component 104 are preferably manufactured from titanium, specifically Ti 6 Al 4 V-ELI, though the same can be manufactured from any other suitable medical grade material, such as stainless steel, cobalt-chromium alloys, titanium allows, zirconium alloys and combinations thereof.

Additionally, the modular head compression screw device 100 and its components can be of any suitable size, shape, and configuration as is known in the art without affecting the overall concept of the invention. One of ordinary skill in the art will appreciate that the shape and size of the modular head compression screw device 100 as shown in FIGS. 1-6 is for illustrative purposes only, and many other shapes and sizes of the modular head compression screw device 100 are well within the scope of the present disclosure. Although dimensions of the modular head compression screw device 100 (i.e., length, width, and height) are important design parameters for good performance, the modular head compression screw device 100 may be of any shape, size or configuration that ensures optimal performance during use.

As shown in FIGS. 1-6, the cannulated compression screw component 102 of the modular head compression screw device 100 comprises a tubular body 106 having a first end 108 and a second end 110. The tubular body 106 has a tubular wall 103 defining a cavity 114, and the tubular body 106 is cannulated to allow for the removal of bone pieces as the screw is inserted into the bone. The tubular body 106 further includes a first exterior thread 118 portion on an exterior tubular surface portion 120 of the tubular wall 103. This exterior thread 118 extends from the second end 110 of the tubular body 106. A second exterior thread portion 123 is positioned around the diameter, beginning adjacent to the first end 108 to create a posted compression screw. A third thread portion 112 has a first end 111 which has a diminished thread portion near the first thread portion 118 and a second end 113 having a diminished thread portion. The third thread portion 112 is disposed between the first thread 118 and second thread 123. Further, a modular head component 104 is removably secured at the first end 108 via the second exterior or second thread 123. The modular head component 104 is configured to engage with a driver (not shown) to advance the compression screw into the bone or other material. Finally, the tubular body 106 has a penetrating tip 122 at the second end 110 which includes a recessed area 127 leading to the cannulated opening. The recessed area 127 serves to remove the portion of the bone or other material as the screw is inserted into the bone. The tubular body 106 and the penetrating tip 122 are formed as a single piece.

Additionally, the modular head compression screw device 100 includes a plurality of interchangeable modular head components 104. Each of the modular head components 104 have a threaded head component 124 and a fixation component 126. The threaded head component 124 comprises internal threads (not shown) around its circumference which matingly secures to the first end 108 via the second exterior threads 123 of the tubular body 106. The fixation component 126 acts to engage tendons and/or bones to reduce fractures, compress osteotomies, etc. The fixation component 126 can be positioned radially in any position, and spins independently of the threaded head component 124.

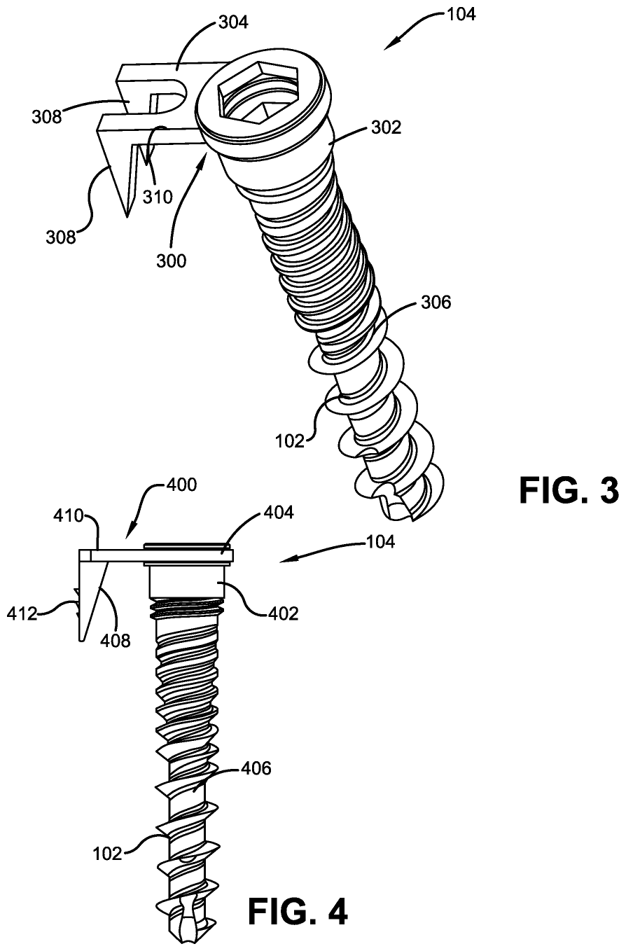

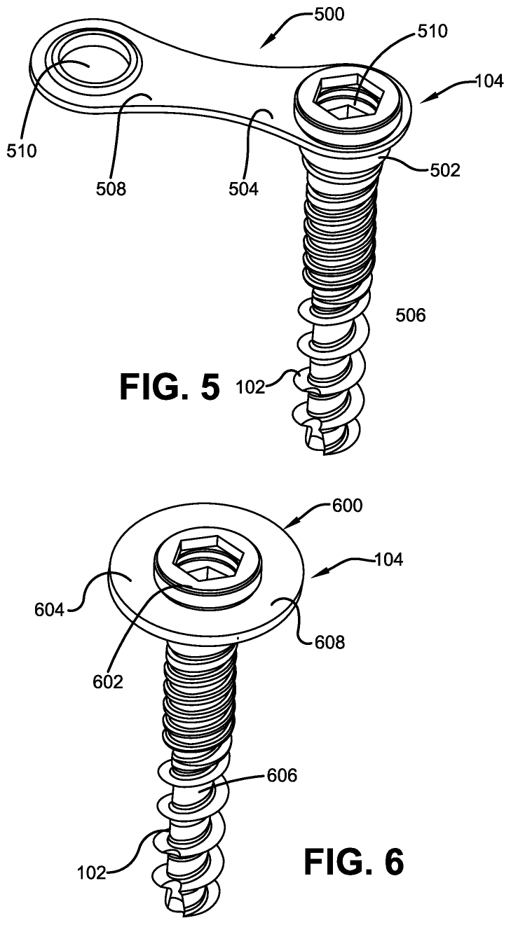

Thus, the modular head compression screw device 100 is designed to fit a plurality of different modular head components 104. The ability to attach different modular head components 104 onto the same tubular body 106 will allow the surgeon to effectively reduce fractures, compress osteotomies and anchor tendons without the need for numerous large surgical sets. Any suitable modular head component 104 can be utilized as is known in the art, depending on the needs or requirements of a user or the particular procedure. However, the present invention discloses five specific modular head components 104. Specifically, the modular head components 104 can comprise a double-winged compression head (see FIG. 2), a hook plate head (see FIG. 3), a single wing compression head (see FIG. 4), a plate head (see FIG. 5) and a simple washer head (see FIG. 6).

As shown in FIG. 2, the modular head component 104 can be a double winged compression head component 200. The double winged compression head component 200 comprises a threaded head component 202 secured to a fixation component 204. The threaded head component 202 is rotatably attached to the fixation component 204 via mating threads or a press fit by pressing the component 204 over the head of the head 200, which allows the fixation component 204 to be positioned radially in any position, and to spin independently of the threaded head component 202. The threaded head component 202 utilizes internal threads (not shown) around its circumference to matingly secure to the tubular body 206 of the compression screw component 102. Specifically, the double-winged compression head component 200 comprises opposing sides 208 that each comprise a wedge-like trocar component 210. Further, each side 208 also comprises anti-backout features (or trocar retention features) 212 which grip into bone to prevent the modular head compression screw 100 from migrating out of the bone. Once inserted, the double-winged compression head component 200 utilizes the wedge-like trocar components 210 of the opposing sides 208 to compress any bone on either side of the double-winged compression head component 200 in towards the center of the modular head compression screw 100.

As shown in FIG. 3, the modular head component 104 can be a hook plate head component 300. The hook plate head component 300 comprises a threaded head component 302 rotatably attached to the fixation component 304 via mating threads or a press fit (not shown), which allows the fixation component 304 to be positioned radially in any position and to spin independently of the threaded head component 302. The threaded head component 302 then utilizes internal threads (not shown) around its circumference to matingly secure to the tubular body 306 of the compression screw component 102. Specifically, the hook plate head component 300 comprises two trocar components 308 positioned on one side 310 of the hook plate head component 300. Once inserted, the hook plate head component 300 fixates the terminal end of a bone. Specifically, the two trocar components 308 grab onto the end of a bone to hold it in place.

As shown in FIG. 4, the modular head component 104 can be a single wing compression head component 400. The single wing compression head component 400 comprises a threaded head component 402 rotatably attached to the fixation component 404 via mating threads or a press fit, which allows the fixation component 404 to be positioned radially in any position and to spin independently of the threaded head component 402. The threaded head component 402 then utilizes internal threads (not shown) around its circumference to matingly secure to the tubular body 406 of the compression screw component 102. Specifically, the single wing compression head component 400 comprises a wedge-like trocar component 408 only on one side 410. The wedge-like trocar component 408 also comprises anti-backout features (or trocar retention features) 412 which grip into bone to prevent the modular head compression screw 100 from loosening or migrating out of the bone. Once inserted, the single wing compression head 400 pulls bone from one side of the compression screw 100 to the center of the compression screw component 102.

As shown in FIG. 5, the modular head component 104 can be a plate head component 500. The plate head component 500 comprises a threaded head component 502 rotatably attached to the fixation component 504 via mating threads or a press fit (not shown), which allows the fixation component 504 to be positioned radially in any position and to spin independently of the threaded-head component 502. The threaded-head component 502 then utilizes internal threads (not shown) around its circumference to matingly secure to the tubular body 506 of the compression screw component 102. Specifically, the plate head component 500 comprises a bone plate 508 with at least two screw holes 510, but the bone plate 508 could have additional screw holes 510 for stability, if needed. Once inserted, the plate head component 500 is affixed on opposite sides of an osteotomy or fracture to stabilize it.

As shown in FIG. 6, the modular head component 104 can be a simple washer head component 600. The simple washer head component 600 comprises a threaded head component 602 rotatably attached to the fixation component 604 via mating threads or a press fit (not shown), which allows the fixation component 604 to be positioned radially in any position and to spin independently of the threaded-head component 602. The threaded head component 602 then utilizes internal threads (not shown) around its circumference to matingly secure to the tubular body 606 of the compression screw component 102. Specifically, the simple washer head component 600 comprises a washer component 608. Once inserted, the simple washer head component 600 adds compression to where the compression screw component 102 is placed. Specifically, compression is added axially down the compression screw component 102. For example, a small fracture could be compressed together by the washer head component 600.

Certain terms are used throughout the following description and claims to refer to particular features or components. As one skilled in the art will appreciate, different persons may refer to the same feature or component by different names. This document does not intend to distinguish between components or features that differ in name but not structure or function.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surgical screw comprising:
a body comprised of a length, a top end, a bottom end and a cavity extending the length of the body;
a first threaded portion disposed at the bottom end of the body;
a second threaded portion disposed at the top end of the body;
a tip provided on the bottom end of the body, wherein the tip is comprised of a recessed area that connects with the cavity.

2. The surgical screw as recited in claim 1, wherein the body further comprises a third threaded portion disposed between the first and second threaded portions.

3. The surgical screw as recited in claim 2, wherein the third threaded portion has a first end and a second end with at least one of the first and second ends having a diminished thread.

4. The surgical screw as recited in claim 3, wherein the surgical screw is comprised of a select one of a titanium, a stainless steel, a cobalt-chromium alloy, a titanium alloy, a zirconium alloy or a combination thereof.

5. The surgical screw as recited in claim 4, wherein the top end of the body comprises a removable compression head.

6. The surgical screw as recited in claim 5, wherein the removable compression head is selected from a group consisting of a double winged compression head, a hook plate head, a single wing compression head, a plate head, a simple washer head and a combination thereof.

7. The surgical screw as recited in claim 6, wherein the removable compression head provides a compression force to an object when the surgical screw is driven into said object.

8. The surgical screw as recited in claim 7, wherein the removable compression head comprises an anti-backout feature.

9. The surgical screw as recited in claim 8, wherein the anti-backout feature includes a plurality of gripping elements for securing to the object.

10. The surgical screw as recited in claim 6, wherein each of the double winged compression head and the single wing compression head is wedge shaped.

11. The surgical screw as recited in claim 9, wherein the cavity is open at the top end of the body.

12. A bone screw comprising:
a body having a top end, a bottom end, and a cavity extending from the top end to the bottom end;
a tip provided at the bottom end of the body, wherein the tip comprises a cutting edge;
a threaded portion provided between the top end and bottom end of the body; and
a winged shaped compression head provided on the top end, wherein the winged shaped compression head is comprised of a plurality of anti-backout elements.

13. The bone screw as recited in claim 12, wherein the winged shaped compression head is wedge shaped.

14. The bone screw as recited in claim 13, wherein the threaded portion is comprised of a first threaded portion and a second threaded portion, and further wherein the first threaded portion differs from the second threaded portion.

15. The bone screw as recited in claim 14, wherein the threaded portion further comprises a third threaded portion distinct from the first and second threaded portions.

16. The bone screw as recited in claim 15, wherein the third threaded portion is comprised of a first end and a second end, and further wherein at least one of the first and second ends have a diminished thread.

17. The bone screw as recited in claim 16, wherein the winged shaped compression head is removable from the body.

18. A surgical bone screw comprising:
a screw body having a top end and a bottom end, with the top end having a compression head;
a cavity contained within the screw body and extending from the compression head to the bottom end;
a plurality of threads provided on an exterior of the screw body;
a penetrating tip provided on the bottom end of the screw body and connecting to the cavity; and
wherein the compression head is removable from the screw body.

19. The surgical bone screw as recited in claim 18, wherein the compression head is selected from a group consisting of a double winged compression head, a hook plate head, a single wing compression head, a plate head, a simple washer head and a combination thereof.

20. The surgical bone screw as recited in claim 18, wherein the compression head includes one of a securing feature or an anti-backout feature, and further wherein the compression head is removable from the screw body.
